(19) United States
(12) Patent Application Publication
(10) Pub. No.: US 2004/0194156 A1
(43) Pub. Date: Sep. 30, 2004

(54) Transgenic aquatic invertebrates as a bioreactor for production of recombinant polypeptides

(75) Inventor: Hao Fan, Bothell, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Califa LLC, Bothell, WA

(21) Appl. No.: 10/745,288

(22) Filed: Dec. 23, 2003

Related U.S. Application Data
(60) Provisional application No. 60/436,875, filed on Dec. 27, 2002.

(51) Int. Cl. A01K 67/033

(52) U.S. Cl. 800/8; 800/13

(57) ABSTRACT

Disclosed are transgenic aquatic invertebrate for use as bioreactors to express heterologous recombinant polypeptides and its application. Also included are methods for producing transgenic Brine species and Brine cysts, and methods for expressing heterologous proteins is said cysts. The method includes introducing the foreign genes into cysts of a Brine shrimp, identification of transgenic individual and the generation of a parthenogenesis culture for transgene expression. The transgenic aquatic invertebrates described will be used as an alternative method for the low cost large-scale production of recombinant proteins, such as therapeutic proteins, polypeptide based vaccine or anti-microbial proteins, proteins for use as a food source in humans, animals and aquaculture.

### BACKGROUND OF THE INVENTION

1. Technical Field

This invention relates generally to transgenic aquatic invertebrates and uses thereof as host systems for the production of recombinant polypeptides.

2. Description of the Related Art

The production of recombinant proteins is one of the major successes of biotechnology. The manufacture of recombinant proteins has been accomplished in several different systems, including bacteria, yeast, baculovirus-infected insect cells, mammalian cells in culture, plant cells, animals and in the organs of transgenic animals, such as cows, wherein the proteins are isolated from milk. Bacterial and yeast cultures are the two most common systems for the production of large quantities of recombinant proteins due to the development of advanced fermentation technologies. However, these techniques are limited by their inability to properly fold and/or post-translationally modify the proteins, which often result in biologically inactive proteins that are not useful as clinical agents. Post-translational modifications of proteins, including phosphorylation, acetylation, amidation, glycosylation and the like, can affect either the activity of the recombinant protein or the lifetime of the protein in circulation.

Baculovirus-infected insect systems and mammalian cells in culture are typically very good systems for expressing heterologous DNA under controlled conditions (Singh et al., J. Interferon Cytokine Res., 16:577-584, 1996; Groner: The Biology of Baculoviruses, R. R. Ganados and B. A. Federici (Eds.), CRC Press, Boca Raton, Fla., pp. 177-202, 1986; Guarino and Summers. J. Virol., 61:2091-2099,1987; U.S. Pat. No. 5,869,33). However, the expression of protein in cultured cells requires very restrictive conditions and equipment that results in exceedingly high costs. Further the increasing concerns of prion and human virus contamination in mammalian and human cell cultures can never been underestimated.

Numerous reports on the production of proteins with transgenic algae, fishes, plants and livestock, such as pigs, sheep, and cows have also been reported (see examples of U.S. Pat. No. 6,027,900; U.S. Pat. No. 6,380,458; U.S. Pat. No. 6,303, U.S. Pat. No. 6,140,552; U.S. Pat. No. 4,736,866; U.S. Pat. No. 6,339,183; Bhandari & Shashidhara, Oncogene 20(47):6871-80, 2001; U.S. patent application Ser. No. 20020013955; U.S. Pat. No. 6,201,167; Muller, W. J., et al., Cell 54:105-115, 1988; Miller, K. F., etal., J. Endocrin. 120:481-488, 1989; Vize, P. D., et al., J. Cell Sci. 90:295-300, 1988; Ebert, K. et aL, Mol. Endocrin. 2:277-283, 1988; Nancarrow, et al., Theriogenology 27:263, 1987; Clark, A. J. et al., Bio/Technology 7:487-482, 1989; Simons, J., et al., Bio/Technology 6:179-183, 1988; Hanover, S. V., et al., Deutche Tierarztliche Wochenschrift 94:476-478, 1987; Simons, J., et al., Bio/Technology 6:179-183, 1988; and Pursel. et al., Science 244:1281-1288, 1989, Chen, et al., Biotechnology Annual Review 2:205-236, 1996). However, although the transgenic animals possess many benefits that make them valuable sources for production of a desired recombinant protein, the maintenance of living transgenic animals, in addition to maintaining variable transgene stocks are extremely labor intensive and costly. Furthermore, the environmental concerns of transgenic plants has yet to be addressed.

Although insect cell culture is widely used for gene expression with baculovirus-based vectors, the development of transgenic Arthropods have primarily focused on insects such as Drosophila (Rubin & Spradling. Science 218: 348-353, 1982), wherein gene expression was mediated with transposons of the P-element and mariner (Lidholm et al., Genetics 134: 859-868, 1993), and Tc1-lie element (Minos Loukeris et al., Science 270: 2002-2005, 1995a; Loukeris et al., Proc Natl Acad Sci USA 92: 9485-9489, 1995b; Presnail & Hoy. Proc Natl Acad Sci USA 89: 7732-7736, 1992; and Odindo. Insect Science Appl 9: 399-404) achieved via transformation with direct injection of DNA into the ovaries through the mother's body. Once again, the costs associated with the generation and maintenance of such transgenic organisms makes the large-scale use of these systems for the production of proteins difficult.

In addition to the production of therapeutic, immunogenic, and anti-microbial proteins, there is also a need for producing protein sources for consumption. Humans require a supply of essential amino acids for a well-balanced metabolism. The body cannot synthesize these amino acids by itself and, therefore, suitable quantities of these amino acids must be taken in by way of balanced nutrition. Nutritional deficiency in one or more of the essential amino acids leads to metabolic disturbances such as hyperlipidemia, diabetes mellitus, hypertension, and weight problems. Therefore, there remains a need in the art for providing a food source high in essential amino acids, which also provides proteins that can be advantageous to the health of the recipient.

Accordingly, there is a need in the art for cost effective and less labor-intensive methods for the production of recombinant proteins.

### BRIEF SUMMARY OF THE INVENTION

This invention relates primarily to the use of transgenic aquatic invertebrates as a host system for the production of recombinant polypeptides and proteins. Transformation methods, promoters and vector construction are described. Further, methods for the detection of a transgenic aquatic invertebrates and their potential application are also described.

The present invention provides a transgenic aquatic invertebrate comprising a polynucleotide sequence that encodes a heterologous protein, polypeptide or peptide.

The present invention further provides a transgenic aquatic invertebrate comprising a polynucleotide sequence that encodes a heterologous polypeptide or peptide, wherein the transgenic aquatic invertebrate is not algae.

In a related aspect, the transgenic aquatic invertebrate of the present invention is a crustacean.

In yet another embodiment, the transgenic aquatic invertebrate of the present invention is selected from isopods, copepods, branchiopods, and decapods.

In a related embodiment, the branchiopods of the present invention are fairy shrimp, Brine shrimp, daphnia, clam shrimp and tadpole shrimp.

In yet another related embodiment, the transgenic aquatic invertebrates of the present invention are crabs, lobsters, crayfish, and shrimp.

In yet another aspect of the present invention, the transgenic aquatic invertebrates from the Artemia species of shrimp.

One aspect of the present invention provides for a transgenic aquatic invertebrate that comprises a polynucleotide sequence wherein said sequence encodes for a therapeutic peptide or polypeptide that may be any peptide or polypeptide that has desirable therapeutic properties. With respect to therapeutic polypeptides or peptides, these may include, without limitation therapeutic peptides and polypeptides selected from the group of polypeptides consisting of human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1, IL-2, IL-5, IL10, IL-11, IL-12, IL-18, tumor necrosis factor, colony stimulating factor, GM-CSF, and human cholesterol hydrolase.

In another aspect the present invention provides for a transgenic aquatic invertebrate that comprises a polynucleotide sequence wherein said sequence encodes for an immunogenic polypeptide or peptide. Such an immunogenic polypeptide or peptide may comprise any desirous polypeptide. For example, immunogenic peptides or polypeptides may be derived from viruses, bacteria, cancers, parasites, prions, fungi, or any polypeptide against which the development an immune response, be it either a B cell or a T cell response, would be advantageous. For example, these polypeptides and peptides include, without limitation, polypeptides and peptides derived from proteins of influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In a related aspect, the present invention provides for a transgenic aquatic invertebrate that comprises a polynucleotide sequence wherein said sequence encodes at least an antigen-binding fragment of an antibody. In a related aspect, the polynucleotide may encode an immunoglobulin heavy chain having at least an antigen-binding domain. In a related aspect of the present invention, the transgenic aquatic invertebrate further comprises a polynucleotide sequence encoding an immunoglobulin light chain having at least an antigen-binding domain.

In another aspect the present invention provides for a transgenic aquatic invertebrate that comprises a polynucleotide sequence wherein said sequence encodes for an anti-microbial polypeptide or peptide. Such anti-microbial polypeptides, peptides, or proteins may be derived from either “normal flora”, which are microbes that live within limited areas of the body, such as the skin, mouth, large intestine, small intestine, stomach and vagina, or the anti-microbial proteins may be derived from pathogens. For example, these anti-microbial polypeptides and peptides include, without limitation, anti-microbial proteins selected from the group consisting of protegrin, magainins, dermaseptins, PGLa peptides, XPF peptides, adrenoregulins, BPI protein, BPI peptides, caeruleins, performs, insect defensins, insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

In yet another aspect, the present invention provides for Artemia shrimp or fairy shrimp that comprise a polynucleotide sequence that encodes a heterologous polypeptide or peptide.

In a related aspect, the present invention provides an Artemia or fairy shrimp comprising a polynucleotide sequence encoding a therapeutic peptide or polypeptide that has desirable therapeutic effects. With respect to therapeutic polypeptides or peptides, these may include, without limitation, a therapeutic polypeptide or peptide selected from the group consisting of human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors Vil and IX, thrombopoietin, alpha-antitrypsin, erthropoietin, urokinase, IFN-gamma, IL-1, IL-2, IL-5, IL10, IL-11, IL-12, IL-18, tumor necrosis factor,

In another aspect, the present invention provides for an Artemia or fairy shrimp comprising a polynucleotide sequence encoding an immunogenic polypeptide or peptide. Such an immunogenic polypeptide or peptide may comprise any desirous polypeptide or peptide. For example, immunogenic polypeptides and peptides may be derived from viruses, bacteria, cancers, parasites, prions, fungi, or any polypeptide against which the development of an immune response, be it either a B cell or T cell response, would be advantageous. For example, these polypeptides and peptides include, without limitation those derived from proteins associated with influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In a further embodiment, the present invention provides for Artemia or fairy shrimp comprising a polynucleotide sequence wherein said sequence encodes at least an antigen-binding fragment of an antibody. In a related aspect, the polynucleotide sequence may encode an immunoglobulin heavy chain having at least an antigen-binding domain. In a related aspect of the present invention, the Artemia shrimp further comprises a polynucleotide sequence encoding an immunoglobulin light chain having at least an antigen-binding domain.

In another aspect the present invention provides for Artemia or fairy shrimp that comprise a polynucleotide sequence wherein said sequence encodes for an anti-microbial polypeptide or peptide. Such anti-microbial polypeptides or peptides may be derived from either “normal flora”, which are microbes that live in areas of the body such as the skin, mouth, large intestine, and vagina, or the anti-microbial proteins may be derived from pathogens. For example, these anti-microbial proteins may be derived from protegrins, magainins, dermaseptins, PGLa peptides, XPF peptides, adrenoregulins, BPI protein, BPI peptides, caeruleins, performs, insect defensins,insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

In another aspect, the present invention provides an aquatic invertebrate bioreactor for the production of a heterologous polypeptide or peptide, said bioreactor comprising an aquatic invertebrate comprising a polynucleotide sequence, wherein said polynucleotide sequence encodes a heterologous polypeptide or peptide.

In a related aspect, the present invention provides an aquatic invertebrate bioreactor, wherein the aquatic invertebrate is a crustacean. In yet another aspect of the present invention provides an aquatic invertebrate bioreactor, wherein the aquatic invertebrate is selected from the group consisting of isopods, copepods, branchiopods, and decapods. In a related embodiment, the branchiopods of the present invention are fairy shrimp, Brine shrimp, daphnia, clam shrimp and tadpole shrimp. In a related aspect of the present invention, the decapods include crabs, lobsters, crayfish and shrimp. In a related aspect, the present invention provides an aquatic invertebrate bioreactor, wherein the aquatic invertebrate is an Artemia or fairy shrimp.

In yet another embodiment, the present invention provides for an invertebrate bioreactor for the production of a heterologous polypeptide or peptide, wherein the polypeptide or peptide is a therapeutic peptide or polypeptide that has desirable therapeutic effects. With respect to therapeutic polypeptides or peptides, these may include, without limitation the therapeutic polypeptides or peptides selected from the group consisting of human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1, IL-2, IL-5, IL10, IL-11, IL-12, IL-18, tumor necrosis factor, colony stimulating factor, GM-CSF, and human cholesterol hydrolase.

In yet another embodiment, the present invention provides for an aquatic invertebrate bioreactor for the production of a heterologous polypeptide or peptide, wherein the protein is an immunogenic polypeptide or peptide. Such an immunogenic polypeptide or peptide may comprise any desirous polypeptide or peptide. For example, immunogenic polypeptides or peptides may be derived from viruses, bacteria, cancers, parasites, prions, fungi, or any polypeptide against which the development of a an immune response, be it either a B cell or T cell response, would be advantageous. For example, these polypeptides or peptides include, without limitation polypeptides and peptides derived from proteins associated with influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In a further embodiment, the present invention provides for an aquatic invertebrate bioreactor comprising a polynucleotide sequence wherein said sequence encodes at least an antigen-binding fragment of an antibody. In a related aspect, the polynucleotide sequence may encode an immunoglobulin heavy chain having at least an antigen-binding domain. In a related aspect of the present invention, the aquatic invertebrate bioreactor further comprises a polynucleotide sequence encoding an immunoglobulin light chain having at least an antigen-binding domain.

In another aspect the present invention provides for an aquatic invertebrate bioreactor that comprise a polynucleotide sequence wherein said sequence encodes for an anti-microbial polypeptide or peptide. Such anti-microbial polypeptides or peptides may be derived from either “normal flora”, which are microbes that live with areas of the body such as the skin, mouth, large intestine, and vagina, or the anti-microbial proteins may be derived from pathogens. For example, these anti-microbial proteins may be derived from protegrins, magainins, dermaseptins, PGLa peptides, XPF peptides, adrenoregulins, BPI protein, BPI peptides, caeruleins, performs, insect defensins, insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

Another aspect of the present invention provides for methods of producing a heterologous protein in an aquatic invertebrate comprising the steps of: producing a construct comprising a polynucleotide sequence encoding for a heterologous polypeptide or peptide, and inserting said construct into the genome of an aquatic invertebrate such that the expression of said polynucleotide sequence encoding said heterologous polypeptide or peptide is under the control of a regulatory region of DNA that regulates the expression of said heterologous polypeptide or peptide.

In a related aspect, the present invention provides methods for producing a heterologous polypeptide or peptide in a crustacean. In yet another related aspect, the present invention provides methods for producing a heterologous polypeptide or peptide in isopods, copepods, branchiopods, and decapods. In a related embodiment, the branchiopods of the present invention are fairy shrimp, Brine shrimp, daphnia, clam shrimp and tadpole shrimp. In yet another related aspect, the present invention provides methods for producing a heterologous polypeptide or peptide in an Artemia or fairy shrimp.

In a related aspect, the present invention provides methods for producing a therapeutic polypeptide or peptide in a crustacean. In yet another related aspect, the present invention provides methods for producing a therapeutic polypeptides or peptides in isopods, copepods, branchiopods, and decapods. In a related embodiment, the present invention provides methods for producing a therapeutic protein in a branchiopod, wherein the branchiopod of the present invention is selected from fairy shrimp, Brine shrimp, daphnia, clam shrimp and tadpole shrimp.

In yet another related aspect, the present invention provides methods for producing a therapeutic polypeptide or peptide in an Artemia or fairy shrimp. In a related aspect of the present invention, the therapeutic polypeptides or peptides produced may be any therapeutic polypeptide or peptide that has desirable therapeutic effects. With respect to therapeutic polypeptides or peptides, these may include, without limitation human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1, IL-2, IL-5, IL10, IL-11, IL-12, IL-18, tumor necrosis factor, colony stimulating factor, GM-CSF, and human cholesterol hydrolase.

In a related aspect, the present invention provides methods for producing an immunogenic polypeptide or peptide in a crustacean. In yet another related aspect, the present invention provides methods for producing an immunogenic polypeptide or peptide in isopods, copepods, and decapods. In yet another embodiment, the present invention provides methods for producing an immunogenic polypeptide or peptide in a branchiopod, wherein the branchiopod is selected from the group consisting of fairy shrimp, Brine shrimp, daphnia, clam shrimp and tadpole shrimp.

In yet another related aspect, the present invention provides methods for producing an immunogenic polypeptide or peptide in an Artemia or fairy shrimp. Such an immunogenic polypeptide or peptide may comprise any desirous polypeptide or peptide. For example, immunogenic polypeptides or peptides may be derived from viruses, bacteria, cancers, parasites, prions, fungi, or any polypeptide against which the development of a an immune response, be it either a B cell or T cell response, would be advantageous. For example, these polypeptides include, without limitation polypeptides derived from proteins associated with influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In a related aspect, the present invention provides methods for producing at least an antigen-binding fragment of an antibody in a crustacean. In a related aspect, the present invention provides methods for producing an immunoglobulin heavy chain having at least an antigen-binding fragment in a crustacean. In a related aspect, the present invention provides methods for producing an antigen-binding fragment of an antibody in isopods, copepods, branchiopods, and decapods. In yet another embodiment, the present invention provides methods for producing an immunoglobulin heavy chain having at least an antigen binding fragment in a branchiopod, wherein the branchiopod is selected from the group consisting of fairy shrimp, Brine shrimp, daphnia, clam shrimp and tadpole shrimp. In yet another related aspect, the present invention provides methods for producing an immunoglobulin heavy chain having at least an antigen-binding fragment in isopods, copepods, branchiopods, and decapods. In another aspect, the present invention provides methods for producing at least an antigen binding fragment of an antibody in a branchiopod, wherein the branchiopod is selected from the group consisting of fairy shrimp, Brine shrimp, daphnia, clam shrimp and tadpole shrimp. Another aspect of the present invention provides methods for producing at least an antigen-binding fragment of an antibody in an Artemia shrimp. In yet another related aspect, the present invention provides methods for producing an immunoglobulin heavy chain having at least an antigen-binding fragment in an Artemia shrimp. In a related embodiment, the crustacean further comprises an immunoglobulin light chain having at least an antigen-binding domain. In yet a further aspect, the isopods, copepods, branchiopods, and decapods further comprise an immunoglobulin light chain having at least an antigen-binding domain. In a related embodiment, the branchiopod, wherein the branchiopod is selected from the group consisting of fairy shrimp, Brine shrimp, daphnia, clam shrimp and tadpole shrimp, further comprise an immunoglobulin light chain having at least an antigen-binding domain. In yet another related aspect, the Artemia or fairy shrimp of the present invention further comprise an immunoglobulin light chain having at least an antigen-binding domain.

In a related aspect, the present invention provides methods for producing an anti-microbial polypeptide or peptide in a crustacean. In yet another related aspect, the present invention provides methods for producing an anti-microbial polypeptide or peptide in isopods, copepods, branchiopods, and decapods. In yet another embodiment, the present invention provides methods for producing an anti-microbial polypeptide or peptide in a branchiopod, wherein the branchiopod is selected from the group consisting of fairy shrimp, Brine shrimp, daphnia, clam shrimp and tadpole shrimp. In yet another related aspect, the present invention provides methods for producing an anti-microbial polypeptide or peptide in an Artemia.

In a further embodiment, the present invention provides methods for producing an Artemia shrimp comprising a polynucleotide sequence encoding a heterologous polypeptide or peptide comprising the steps of: hydrating the cysts; decapsulating the cysts; producing a construct comprising the polynucleotide sequence encoding for a heterologous polypeptide or peptide; and inserting the construct into the genome of the Artemia shrimp such that the expression of said polynucleotide encoding said heterologous polypeptide or peptide is under the control of a regulatory region of DNA that regulates the expression of said heterologous polypeptide or peptide.

In another embodiment, the present invention provides for methods of manufacturing an aquatic invertebrate comprising a polynucleotide sequence wherein said sequence encodes a heterologous polypeptide or peptide comprising the steps of: producing a construct comprising a polynucleotide sequence encoding a heterologous polypeptide or peptide; and inserting said construct into the genome of an aquatic invertebrate such that the expression of said polynucleotide sequence encoding said heterologous polypeptide or peptide is under the control of a regulatory region of DNA that regulates the expression of said heterologous polypeptide or peptide.

In yet another embodiment, the present invention provides methods for the manufacture of an Artemia cyst comprising a polynucleotide encoding a heterologous polypeptide or peptide wherein said sequence encodes a heterologous polypeptide or peptide comprising the steps of: hydrating said cysts; decapsulating said hydrated cyst; producing a construct comprising said polynucleotide sequence encoding said heterologous polypeptide or peptide; and inserting said construct into the genome of a Artemia shrimp such that the expression of said polynucleotide sequence encoding the heterologous polypeptide or peptide is under the control of a regulatory region of DNA that regulates the expression of said heterologous polypeptide or peptide.

In yet another embodiment, the present invention provides kits for producing heterologous polypeptide or peptide comprising an Artemia cyst comprising a polynucleotide sequence encoding a heterologous polypeptide or peptide; and instructions for using the same to produce a heterologous polypeptide or peptide.

In still another embodiment, the present invention provides a kit for producing a therapeutic polypeptide or peptide comprising; an Artemia cyst comprising a polynucleotide sequence encoding a therapeutic polypeptide or peptide; and instructions for using the same to produce a therapeutic polypeptide or peptide. In a related aspect of the present invention, the therapeutic polypeptide or peptide produced by the kit may be any polypeptide or peptide that has desirable therapeutic effects. With respect to the therapeutic polypeptides or peptides capable of being produced by the kit, these may include, but are not limited to human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1, IL-2, IL-5, IL10, IL-11, IL-12, IL-18, tumor necrosis factor, colony stimulating factor, GM-CSF, and human cholesterol hydrolase.

In yet another embodiment, the present invention provides a kit for producing an immunogenic polypeptide or peptide comprising; an Artemia cyst comprising a polynucleotide sequence encoding an immunogenic polypeptide or peptide; and instructions for using the same to produce an immunogenic polypeptide or peptide. Such an immunogenic polypeptide or peptide may comprise any desirous polypeptide or peptide. For example, immunogenic polypeptides or peptides may be derived from viruses, bacteria, cancers, parasites, prions, fungi, or any polypeptide or peptide against which the development of a an immune response, be it either a B cell or T cell response, would be advantageous. For example, these polypeptides or peptides include, without limitation polypeptides derived from proteins associated with influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In yet another embodiment, the present invention provides a kit for producing an anti-microbial polypeptide or peptide comprising: an Artemia cyst comprising a polynucleotide sequence encoding an anti-microbial polypeptide or peptide; and instructions for using the same to produce an anti-microbial polypeptide or peptide. Such anti-microbial polypeptides or peptides may be isolated from animal or human body such as the skin, mouth, large intestine, and vagina, or the anti-microbial polypeptides or peptides may be derived from any source be they plant, fungal, bacterial, etc. For example, these anti-microbial polypeptides or peptides may be derived from protegrins, magainins, dermaseptins, PGLa peptides, XPF peptides, adrenoregulins, BPI protein, BPI peptides, caeruleins, performs, insect defensins, insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

In yet another embodiment, the present invention provides methods for generating an Artemia cyst comprising growing said Artemia shrimp under conditions suitable for inducing said Artemia shrimp to produce resting cysts. In a related embodiment, the conditions for inducing said Artemia shrimp to produce resting cysts include, but are not limited to high salinity (e.g., in one embodiment higher than 6.5% of NaCl), low oxygen levels (e.g., less than 2mg/ml), and chronic food shortages.

In another aspect, the present invention provides a method for the manufacture of a therapeutic polypeptide or peptide comprising the steps of: producing a construct comprising a polynucleotide sequence encoding for a therapeutic polypeptide or peptide; inserting said construct into the genome of an aquatic invertebrate such that the expression of said polynucleotide sequence encoding said therapeutic polypeptide or peptide is under the control of a regulatory region of DNA that regulates the expression of said therapeutic polypeptide or peptide. In a related aspect of the present invention, the therapeutic polypeptide or peptide may be any polypeptide or peptide that has desirable therapeutic effects. With respect to therapeutic polypeptides or peptides, these may include, without limitation human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1,IL-2, IL-5, IL10, IL-11, IL-12, IL-18, tumor necrosis factor, colony stimulating factor, GM-CSF, and human cholesterol hydrolase.

In another embodiment, the present invention provides methods for the manufacture of an immunogenic polypeptide or peptide comprising the steps of: producing a construct comprising a polynucleotide sequence encoding for a immunogenic polypeptide or peptide; inserting said construct into the genome of an aquatic invertebrate such that the expression of said polynucleotide sequence encoding said immunogenic polypeptide or peptide is under the control of a regulatory region of DNA that regulates the expression of said immunogenic polypeptide or peptide. Such an immunogenic polypeptide or peptide may comprise any desirous polypeptide or peptide. For example, immunogenic polypeptides or peptides may be derived from viruses, bacteria, cancers, parasites, prions, fungi, or any polypeptide against which the development of a an immune response, be it either a B cell or T cell response, would be advantageous. For example, these polypeptides or peptides include, without limitation polypeptides and peptides derived from proteins associated with influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In another aspect, the present invention provides a method for the manufacture of an anti-microbial polypeptide or peptide comprising the steps of: producing a construct comprising a polynucleotide sequence encoding for an anti-microbial polypeptide or peptide; inserting said construct into the genome of an aquatic invertebrate such that the expression of said polynucleotide sequence encoding said anti-microbial polypeptide or peptide is under the control of a regulatory region of DNA that regulates the expression of said anti-microbial polypeptide or peptide. Such anti-microbial polypeptides or peptides may be isolated from animal or human body such as the skin, mouth, large intestine, and vagina or the anti-microbial polypeptides or peptides may be derived from any source be they plant, fungal, bacterial, etc. For example, these anti-microbial polypeptides or peptides may be derived protegrins, magainins, dermaseptins, PGLa peptides, XPF peptides, adrenoregulins, BPI protein, BPI peptides, caeruleins, performs, insect defensins, insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

In a related embodiment, the present invention provides methods for the manufacture of a heterologous polypeptide or peptide in an aquatic invertebrate, comprising providing an aquatic invertebrate wherein at least one cell of said invertebrate expresses said polypeptide or peptide, and growing said invertebrate under conditions suitable for the growth and expression of said heterologous polypeptide or peptide. In a related embodiment, the aquatic invertebrate is a crustacean. In yet another related embodiment, the aquatic marine invertebrate is selected from the group consisting of isopods, decapods, branchiopods, and copepods. In another embodiment, the aquatic invertebrate is selected from the group consisting of fairy shrimp, Brine shrimp, daphnia, clam shrimp and tadpole shrimp. In still another aspect, the aquatic invertebrate is selected from the group consisting of crabs, lobsters, crayfish, and shrimp. In yet another aspect, the aquatic invertebrate is an Artemia shrimp.

In a related embodiment, the heterologous polynucleotide encodes a therapeutic polypeptide or peptide. With respect to therapeutic polypeptides or peptides, these may include, without limitation human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1, IL-2, IL-5, IL10, IL-11, IL-12, IL-18, tumor necrosis factor, colony stimulating factor, GM-CSF, and human cholesterol hydrolase.

In a related embodiment, the present invention provides methods for the manufacture of an immunogenic polypeptide or peptide in an aquatic invertebrate, comprising providing an aquatic invertebrate wherein at least one cell of said invertebrate expresses said polypeptide or peptide, and growing said invertebrate under conditions suitable for the growth and expression of said heterologous polypeptide or peptide. For example, these polypeptides or peptides include, without limitation polypeptides and peptides derived from proteins associated with influenza type A, influenza type B, influenza type C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, and other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

In a related embodiment, the heterologous polynucleotide encodes an anti-microbial polypeptide or peptide. For example, these anti-microbial polypeptides or peptides may be derived protegrins, magainins, dermaseptins, PGLa peptides, XPF peptides, adrenoregulins, BPI protein, BPI peptides, caeruleins, performs, insect defensins, insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

In a further embodiment, the present invention provides the heterologous polynucleotide encodes at least an antigen-binding fragment of an antibody. In a related aspect, the polynucleotide sequence may encode an immunoglobulin heavy chain having at least an antigen-binding domain. In a related aspect of the present invention, the Artemia shrimp further comprises a polynucleotide sequence encoding an immunoglobulin light chain having at least an antigen-binding domain.

In yet another embodiment, the present invention provides for a dietetic composition comprising a transgenic aquatic invertebrate comprising a polynucleotide sequence wherein said polynucleotide sequence encodes a heterologous protein, wherein said heterologous protein is suitable for consumption. In a related embodiment, the transgenic aquatic invertebrate is a crustacean. In yet another embodiment, the transgenic aquatic invertebrate is selected from the group consisting of isopods, copepods, branchiopods, and decapods. In a further embodiment, the transgenic aquatic marine invertebrate is selected from the group consisting of fairy shrimp, Brine shrimp, daphnia, clam shrimp and tadpole shrimp. In a further embodiment, the transgenic aquatic invertebrate is selected from crabs, lobsters and shrimp. In a further aspect, the present invention provides for an aquatic invertebrate, wherein said aquatic invertebrate is an Artemia shrimp or a fairy shrimp.

In yet another embodiment, the present invention provides for a snack food comprising a transgenic aquatic invertebrate comprising a polynucleotide sequence wherein said polynucleotide sequence encodes a heterologous protein, wherein said heterologous protein is suitable for consumption. In a related embodiment, said snack food may also comprise an added flavoring, wherein said flavorings are encoded by a transgene, or said flavorings are exogenously added. In a related embodiment, the transgenic aquatic invertebrate is a crustacean. In yet another embodiment, the transgenic aquatic invertebrate is selected from the group consisting of isopods, copepods, branchiopods, and decapods. In a further embodiment the transgenic marine invertebrate is selected from the group consisting of fairy shrimp, Brine shrimp, daphnia, clam shrimp and tadpole shrimp. In a further embodiment, the transgenic aquatic invertebrate is selected from crabs, lobsters and shrimp. In a further aspect, the present invention provides for an aquatic invertebrate, wherein said aquatic invertebrate is an Artemia shrimp or a fairy shrimp.

In another embodiment, the present invention provides transgenic aquatic invertebrates comprising a polynucleotide sequence which encodes a protein suitable as a food source. In a related embodiment the food source can be used for mammals.

### DETAILED DESCRIPTION OF THE INVENTION

The present invention is directed generally to transgenic aquatic invertebrates and their use as a bioreactor for the production of polypeptides, peptides and proteins.

Prior to setting forth the invention, it may be helpful to an understanding thereof to set forth definitions of certain terms that will be used herein.

Definitions

As used herein a “transgene” refers to a heterologous gene or nucleotide sequence encoding a peptide or polypeptide transplanted into an organism, making the recipient a transgenic organism.

As used herein an “aquatic invertebrate” is an aquatic animal without a backbone.

As used herein the term “aquatic” refers to invertebrates from either fresh or salt water.

As used herein “transgenic aquatic invertebrates” of the invention refer to aquatic invertebrates containing a transgene.

As used herein a “heterologous protein” is a protein, polypeptide or peptide that originates in a foreign species, or, in the same species if modified from its original form.

As used herein the terms “polypeptide”, “peptide” and “protein” are used interchangeably to refer to a linear polymer of amino acids linked together by peptide bonds in a specific sequence.

As used herein “therapeutic polypeptides or peptides” are polypeptides or peptides useful in the treatment, remediation, or prevention of a disease or disorder. A “therapeutic protein” also refers to any peptide, polypeptide, or protein that has the capacity to promote wound healing, tissue repair, or tissue regeneration. A therapeutic protein also includes any other peptide, polypeptide, or protein that treats, prevents, or lessens the symptoms or prognosis of any clinical disease, disorder or related biological manifestation.

As used herein the term “construct” refers to a package of genetic material containing more than one element.

As used herein an “immunogenic polypeptide or peptide” is a polypeptide or peptide having the ability to evoke B and/or T cell mediated immune reactions.

As used herein “immunoglobulins” are antibodies, including IgG, IgM, IgA, IgE, and IgD.

As used herein “anti-microbial polypeptides or peptides” refer to any protein that either destroys, prevents the multiplication or growth, or prevents pathogenic action of a microbe, and these polypeptides or peptides are typically derived from animal or human tissues, or any organism or plant that has the desired polypeptide or peptides.

As used herein the term “microbe” refers to any micro-organism that can be pathogenic in nature.

As used herein a “cyst” or “egg” refers to a dormant egg or cyst produced by an aquatic invertebrate in response to certain conditions. A hard outer shell surrounds the cysts.

As used herein an “aquatic invertebrate bioreactor” is a transgenic aquatic organism engineered to produce heterologous polypeptides or peptides.

As used herein a cyst is “hydrated” when they become spherical in shape.

As used herein “decapsulating” is the process whereby the outer hard layer of the Artemia shrimp is removed.

As used herein the term “food source” refers to any polypeptide or peptide generated by the transgenic aquatic invertebrate that is not toxic to animals including humans.

As used herein, “conditions suitable for inducing Artemia shrimp to produce cysts” is any condition that induces stress in the Artemia shrimp, resulting in the shrimp producing cysts.

As used herein, “promoter” is a segment of DNA or RNA of specific sequence, which controls transcription of the functionally linked DNA or RNA. The specific sequence of the promoter allows for RNA polymerase recognition, binding, and initiation of transcription. The promoter may also include further cis acting or trans acting factors that regulate transcription. The promoter may be constitutive or regulated (inducible) by other factors.

The production of transgenic aquatic invertebrates has been limited to transgenic methods for improving the cultivation of these species. To date, no transgenic Brine shrimp has been reported. Further, the ability of such species to act as bioreactors has gone unrecognized. The development of transgenic aquatic invertebrates for the purposes of high-level protein production has heretofore not been described. The focus of this application, the production of transgenic aquatic invertebrates expressing recombinant polypeptides or peptides for therapeutic and other uses, is therefore unique and in many aspects. The production of recombinant polypeptides and peptides in aquatic invertebrates has several advantages over methods employing other transgenic hosts. For example, aquatic invertebrates, such as the Brine shrimps or fairy shrimps are able to produce large numbers of offspring in a relatively short period of time, allowing for the rapid amplification of the heterologous protein of interest. Further, many aquatic invertebrates, such as Brine shrimps and fairy shrimps are able to produce dormant eggs or cysts, which are able to be stored under a wide range of conditions and for extended periods of time, allowing for a readily available source of material from which to produce any desired protein.

Transqenic Aquatic Invertebrates

Aquatic invertebrates of the present invention include, but are not limited to, Coelenterata (jellyfishes and sea anemones), Platyhelminthes (flatworms), Aschelminthes (rotifers, horsehair worms, and roundworms), Mollusca (snails, bivalves, squids, and octopuses), Annelida (segmented worms), Arthropoda (horseshoe crabs, spiders, crabs, centipedes, millipedes, and insects), and Echinodermata (starfishes and sea urchins). The subphyla Urochordata (tunicates) and Cephalochordata (sea lancelets) within the phylum Chordata are also considered invertebrates. Many phyla, such as Ctenophora (comb jellies), Acanthocephala (spiny-headed worms), Brachiopoda (lamp shells), Ectoprocta (bryozoans), and Tardigrada (water bears), are also included among the invertebrates.

A major group of tiny crustaceans, with more than 7,500 species, are the copepods (class Copepoda). These invertebrates are cylindrical, have numerous specialized appendages, and are usually bottom-dealers or zooplankton that are floating animals in which any form of locomotion is weak or absent. Some copepods eat phytoplankton, or floating algae, and are a major food source of many higher marine organisms, including certain species of whales. Most of the parasitic crustaceans are copepods. Parasitic forms commonly attach themselves to fishes and are referred to as fish lice.

Barnacles (class Cirripedia) are marine crustaceans in which the adults are sessile, or remain in one spot, throughout their lives. They may attach to any solid surface including docks, the underwater parts of ships, and even turtles and whales. Some of the approximately 900 species of barnacles are parasites on marine organisms.

Another group of crustaceans, the order Isopoda (class Malacostraca), has mostly marine forms but also include the land-living sow bugs and pill bugs, or wood lice. Isopods are flat and usually gray. A trait of some terrestrial isopods is that they tuck themselves into a ball shape for protection. About 4,000 species of isopods are known.

A related group is the order Amphipoda, also primarily marine, of which there are about 5,500 species. Amphipods are compressed in appearance and look similar to small shrimp.

Another related group is the order Decapoda (class Malacostraca), which include, but are not limited to, species such as crayfish, shrimps, crabs.

Further examples of aquatic invertebrates include, but are not limited to, water fleas (Cyclops v iridis), fairy shrimp (Eubranchipus vernalis), and Brine shrimp (class Branchiopoda). Daphnia sp., Crayfish (genera Astacus and Cambarus), lobsters, shrimps (genus Penaeus), crabs , e.g. Dungeness crabs (Cancer magister), king crabs (Paralithodes camtschatica), blue crab (Callinectes sapidus),

Life Cycle of Brine Shrimp

The Brine shrimp, Artemia sp. is an invertebrate belonging to the class crustacea, and Phylum Arthropoda. The Brine shrimp species has been shown to exist worldwide in hypersaline lakes and ponds in which the salt content may be as high as to achieve saturation. A typical example is Artemia salina var. San Francisco which lives in the Great Salt Lakes in the USA and whose cysts (eggs) are commercially available.

The life cycle of Artemia begins by the hatching of dormant cysts that are about 200 micrometers in diameter. These cysts contain an embryo in a metabolically inactive state known as diapause. The cysts are very hardy and can remain dormant for many years if kept dry. Rehydration of the cysts occurs when they are placed in salt water. After 15 to 20 hours in salt water at an optimal temperature of 25 to 30° C. the cyst bursts and the embryo leaves the shell and is released as a free swimming individual known as a nauplius larva. At the first growth stage the naupilus larva is dependent on its yolk reserve to survive. Depending on the water temperature, the larvae remain in this stage for about 12 hours and molt to the second naupli stage, which feeds on microalgea, bacteria and detritus using hair-like structures on the antennae known as setae. The nauplii will grow and progress through 15 molts before reaching adulthood in approximately 14 days. Adult Artemia on average are about 8 mm long, but can reach lengths up to 20 mm. An adult is a 20 times longer, and 500 times greater in biomass than that of the nauplii stage.

Under optimum conditions of food supply, low salinity and sufficient oxygen, fertilized female shrimp may produce eggs that hatch soon after emerging from the ovisac. The female shrimp is able to produce free-swimming nauplii at rate of up to 75 nauplii per day, a process known as ovoviparous reproduction. If conditions are perfect, the female can live as long as 3 months and produce as many as 300 live nauplii or cysts every 4 days. The female shrimp will produce 10-11 broods over an average life cycle of 50 days. Cyst production is induced by under stress conditions of high salinity, food shortages and decreased oxygen level.

Brine shrimp could be easily raised in a laboratory on a scale ranging from a small glass beaker to a commercially available aqua tank. A high-density outdoor growth tank is also technically easy constructed. Therefore, an isolated growth facility that eliminates environmental concerns related to any transgenic Brine shrimp could be easily established with minimal cost. The simplicity of the Brine shrimps life cycle, their easy growth maintenance and large-scale production capacity creates an ideal model for a transgenic bioreactor.

Brine Shrimp Reproduction and Female Only Culture

Reproduction of Brine shrimp can be both sexual and by way of parthenogenesis (female only). Sexual reproduction exists when both male and female populations are abundant. The male Brine shrimp deposits its sperm into the female's uterus through a copulation event. After fertilization, the eggs either develop into free-swimming nauplius larvae in a process known as ovoviviparous reproduction, which are released by the mother, or they are surrounded by a shell that forms a cyst under conditions of stress.

Some of the Artemia species, e.g., Artemia parthenogenica have a female only reproduction cycle (Parthenogenesis), which involves the development of an unfertilized egg into an adult organism. It takes place in a population of the Brine shrimp, when no males can be found (female colony). Since the female parthenogenesis results only in female shrimp, the eventual result is an all female Brine shrimp population, of genetic homology. This parthenogenesis phenomenon allows creation of a homogeneous culture of transgenic Brine shrimp. Accordingly, in one aspect of the present invention, aquatic organisms, such as certain Brine shrimp, that asexually reproduce or have single sex reproduction are utilized.

Selection of female only Brine shrimp is straightforward and is based on male and female morphological differences as well as clear distinguishing characteristics of their genital regions. Artemia shrimp, with their three eyes, is positively phototactic at low light intensities. A light torch can attract the shrimp and allowing easy capture in an aqua screen or Brine shrimp net.

Cysts Production and Transgenic Stocks

Under conditions of stress, such as low oxygen levels (e.g., less than 2 mg/l), chronic food shortages and/or high salinity in the water (e.g., in one embodiment higher than 6.5% of NaCl), Artemia is induced to produce resting cysts or dormant eggs. The cysts can remain dormant for many years as long as they are kept dry. When the cysts are placed back into salt water they are re-hydrated and resume their development. Adult Brine shrimp can tolerate brief exposures to temperature extremes of −18° C. to +40° C. However, under proper storage condition, Artemia cysts can even tolerant temperatures as low as minus 190° C. and will still hatch when returned to salt water at a normal temperature. Moreover, provided they are fully dry, small proportions will even survive for 2 hours a temperature of 105° C. Artemia cysts are best stored in a tightly sealed container in a cool dry environment, if possible, vacuum packed. This extreme temperature tolerance and long storage shelf life provides feasibility to maintain a large transgene stocks with minimal efforts compared with any other transgenic animals.

Furthermore, adult Artemia shrimp have a non-calcified exoskeleton and up to 63% of their dry weight is composed of protein, which significantly simplifies downstream protein purification and provides one of the highest percentage of protein content to dry weight when compared to other bioreactors.

Gene Transformation

In order to express a heterologous recombinant polypeptide or peptide from DNA in a living system, it is necessary to include sequences that direct the transcription of the gene, the gene sequence encoding the polypeptide or peptide, and sequences that direct the termination of transcription of the gene. Sequences that direct the transcription of the gene are generally in regions located adjacent to the 5′ end of the gene and are termed promoters. Transcription termination signals are located beyond the 3′ end of the coding region of the gene and often contain sequences of MTAAA followed by stretches of variable length comprising pyrimidine rich sequences. Genes to be expressed may be derived from cDNAs produced from mRNA isolated from any biological source expressing the gene of interest, or alternatively, may be derived from genomic DNA isolated from the species of interest. Genomic DNAs are often very large, containing not only the sequences for the gene, but a variable number of introns (intervening sequences removed from RNA transcribed from a gene) as well. There may be some advantage to including introns in genes to be expressed in aquatic invertebrates.

Heterologous DNA, such as that described above, could be introduced in aquatic invertebrates through conventional gene transfer methods such as, Electroporation, DNA micro-injection, Gene gun blast, lipofection and Retro virus transformation (Mol Report Dev 2000 Jun; 56(2 Suppl):281-4 by Tsai HJ.: Mol Mar Biol Biotechnol 1997 Dec; 6(4):289-95 Pantropic retroviral vector integration, expression, and germline transmission in medaka (Oryzias latipes). By Lu JK, Burns JC, Chen TT).

Any established animal transformation methods can be used for the purpose. The preferred method for the transfer of a desired gene into an aquatic invertebrate is electroporation. This is a technique in which the cells or eggs to be electroporated are bathed in a solution of the DNA to be incorporated and an electric current is applied. The current opens the pores in the cysts or cells long enough for the surrounding DNA to enter. The use of this technique requires no special skill on the part of the investigator, and hundreds of potential transgenic aquatic invertebrates can be generated from a single experiment (Inoue et al., Cell Differ. Dev. 29:123-129, 1990).

Methods of producing biologically active molecules by transfer of recombinant genes into cell in culture and into live animals have been developed. For example, DNA molecules have been introduced into cultured cells by calcium phosphate precipitation or electroporation (see for example Graham and Van der Ebb Virology, 52, 456-467, 1973; Perucho et al., Cell, 22, 9-17, 1980; Chu et al. Nucleic Acids Research, 15:1311-1326, 1987; and Bishop and Smith Molecular Biology Medicine, 6, 283-298, 1989). DNA molecules have also been introduced into the nucleus of cells in culture by direct microinjection (see for example Gordon et al. Proc. Natl Acad. Sci. USA, 77, 7380-7384, 1980; Gordon and Ruttel. Methods in Enzymology, 101, 411-433, 1983; and U.S. Pat. No. 4, 873, 191).

Retroviral vectors have also been used to introduce DNA molecules into the genome of animals (see for example Jaenisch et al. Cell, 24, 519, 1981; and Soriano et al., Science, 234, 1409-1413, 1986).

For the class of crustaceans in which gene transformation has been attempted, reports have been limited to those attempting to improve the production levels of cultivated of shrimp (see for example U.S. Pat. No. 5,712,091; Li and Tsai. Mol. Reprod. Dev. 56 (2): 149-54, 2000; and Tseng et al., Theriogenology 54(9): 1421-1432, 2000). Due to the technical difficulties in maintaining transgenic shrimp cultures, so far no applicable transgenic shrimp strain has been established.

Construction of Expression Cassette

An expression cassette for the expression of recombinant proteins in transgenic Aquatic invertebrates will typically contain: a promoter region, the desired nucleic acid sequence to be expressed derived from an isolated cDNA or genomic clone containing introns, and a eukaryotic 3′-untranslated sequence. In some cases, a selection marker gene or reporter gene is co-transferred into the aquatic invertebrates. Another cassette comprising a reporter gene can also be constructed from a head-to-end with a similar promoter and 3′-untranslated region. The cassette can be cloned into a bacteria vector, typically an E. coli vector, e.g. pBluescript, using standard methods including the polymerase chain reaction (PCR) and restriction enzyme digestion. All necessary sequences that allow the stable propagation in bacteria host e.g. antibiotic selection gene, DNA replication origins, partition sequence, and multi-restriction clone sequence, are in the vectors.

Promoter, Enhancer, and 3′ Untranslated Sequence in Expression Cassette

The native promoter from an aquatic invertebrate is most desirable because it is likely to express the transgene at the highest levels. However, many enkaryotic and virus promoters could be used to produce transgenic aquatic invertebrates. Essentially any promoter that is operable may be used. The favorable promoters, include but are not limited to, promoters isolated from Drosophila, and baculovirus infected insect cells, which belong to the class Arthropod.

The genes encoding the heterologous polypeptides or peptides of the present invention may be operatively associated with a variety of different promoter/enhancer elements, which may include but need not be limited to promoter, enhancer, transcription factor binding site and other gene expression regulatory sequences. The expression elements of these vectors may vary in their strength and specificities. Depending on the host/vector system utilized, any one of a number of suitable transcription and translation elements may be used. The promoter may be in the form of the promoter that is naturally associated with the heterologous nucleic acids sequence of interest. Alternatively, the DNA may be positioned under the control of a recombinant or heterologous promoter, i.e., a promoter that is not normally associated with that gene. In any event, the promoter is included as an “operably linked” promoter, which refers to the situation of a promoter in any embodiment of heterologous nucleic acid sequence according to the present invention in such a manner as to influence the expression of the heterologous polypeptide or peptide. For example, tissue specific promoter/enhancer elements, including distinct promoter and enhancer sequences that are derived from different sources and engineered to produce a recombinant promoter/enhancer element, may be used to regulate the expression of the transferred DNA in specific cell types.

Examples of the promoters include, but are not limited to, those isolated from the Drosoplia heat shock protein hsp70, Kupper, actin, baculovirus promoters derived from immediate-early, delayed-early, late and very late, e.g. ie1 (Guarino and Summers, 1987), ieN (ie2; Carson etal., 1991), ie0, DA26, ETL, 35K, 39K (Guarino and Smith, 1991), gp64 (Blissard and Rohrmann, 1989; Whitford et a., 1989), polyhedrin (Hooft van Iddekinge et al., 1983) and p10 (Kuzio et al., 1984) promoters. Other commercially available promoters can also be used for this purpose, including the widely used human cytomegalovirus (CMV) promoter. Adenovirus major late promoter, human mouse leukemia virus (MoMLV), SV40 virus, promoters containing the Rous sarcoma virus long terminal repeat (RSV LTR), mouse metallothienin gene, Xenopus laevis elongation factor 1.alpha, human heat shock protein gene. Fusion promoter with combination of two or more promoters can also be used in the present invention. A Drosophila hsp70 and CMV fusion promoter is a preferred embodiment of the present invention.

Cis acting elements of promoter enhancer, which can be located to enhance transcription from a given promoter, can also be provided in the expression cassettes. Enhancers, which are active in insect cells, are preferred in the present invention. The preferred viral enhancers are baculoviral, and include hr1, hr2, hr3, hr4 and hr5 (Guarino et al., 1986).

The use of recombinant promoters and/or enhancers to achieve protein expression is generally known to those of skill in the art of molecular biology, for example, see Sambrook et al., (1989). The promoters employed may be constitutive, or inducible, and can be used under the appropriate conditions to direct high level or regulated expression of the introduced DNA segment. Expression cassettes typically also contain transcriptional termination sequences including a polyadenylation sequences. This polyadenylation sequence may be selected from any of a variety of polyadenylation signal sequences known in the art. Preferably, the polyadenylation signal sequence is the SV40 early polyadenylation signal sequence, which is commercially available. The construct may also include one or more introns, which can increase levels of expression of the polynucleotide of interest, particularly where the gene is a cDNA. Any of a variety of introns may be used e.g, the human β-globin intron, which is inserted in the construct at a position 5′ to the DNA of interest.

A potential strong promoter from Artemia that may be utilized is the promoter to drive heat inducible p26 protein, which is highly enriched in Artemia cysts.

Reporter Genes and Screening

In addition to a heterologous polypeptide or peptide, a vector may contain a gene whose product can be detected or selected for. As referred to herein, a “reporter or marker” gene is one whose product can be detected, such as by immunodetection, fluorescence, enzyme activity on a chromogenic or fluorescent substrate, and the like, or selected for by growth conditions. Such reporter genes include, without limitation, green fluorescent protein (GFP), 62 -galactosidase, chloramphenicol acetyltransferase (CAT), luciferase, neomycin phosphotransferase, secreted alkaline phosphatase (SEAP), and human growth hormone (HGH). Selectable markers include genetic resistances to drugs, such as ampicillin, neomycin (G418), hygromycin, and the like. However, the present invention is not limited to these markers, as one of ordinary skill in the art could readily envision using any detectable product that allows one to distinguish a cell in which the transgene was introduced and/or expressed. For example, the gene or transgene may be a structural gene that is heterologous or endogenous to the host. If the transgene is endogenous to the host, detection may be done by comparison with a control of untreated cells. Further, as noted above, RNA derived from a transduced gene may be detected by RT-PCR, even absent a phenotypic change in the cell. In addition, any DNA sequence of the vector that is not normally expressed in the cell comprising an internalized ligand displaying genetic package may be directly selected by methods of detecting specific DNA sequences, such as Hirt extraction, PCR, and rolling circle amplificiation (using, e.g., phi29 or exo(−)BST DNA polymerase, etc.), for example. Direct detection of exogenous DNA sequence circumvents the need for expression of a transgene in the host or target cell and allows detection absent a phenotypic or observable change in the cell.

Construction of a Fusion Protein of Interest

The DNA of interest may be inserted into a construct so that the gene product of a polypeptide is expressed as a fusion product, e.g., a fusion protein having a his-tag, flag-tag, thrombin cleavage sequence, a portion N-terminus or C-terminal portion of known proteins. Production of a fusion protein can facilitate identification and purification of cells or cysts expressing the gene products through enzyme-linked immunosorbent assay (ELISA) using an antibody that binds to the fusion protein and affinity purification.

Production of Therapeutic Polypeptides and Peptides

The transgenic Aquatic invertebrates of the present invention are useful in preparation of medically important peptides and polypeptides. Such products include, but are not limited to, human and salmon calcitonin for osteoporosis, human insulin, growth hormones, growth hormone releasing factor, animal growth hormones, somatostatin, thyrotropin, tissue-type plasminogen activator, and vasopressin, human cholesterol hydrolase, lipocortin, coagulation factors VIII and IX, thrombopoietin , α-1 antitrypsin, erythropoietin, and urokinase. Cytokines include interferons (IF-γ, IF-α, IF-β), interleukins (IL-1), IL-2, IL-5, IL-10, IL-11, IL-12, IL-18), tumor necrosis factors-a (TNF-a), colony stimulating factors, GM-CSF (granulocyte macrophage colony stimulating factor) and human cholesterol hydrolase. Therapeutic polypeptides could be further purified with conventional methods to generate pharmaceutical grade polypeptides to use under different administration routes such as oral, intravenous, subcutaneous, intranasal, intrabronchial or rectal administration.

Production of Immunogenic Polypeptides and Peptides

The transgenic aquatic invertebrates of the present invention are useful in preparation of immunogenic polypeptides and peptides. Immunogenic polypeptides and peptides of the present invention are defined as those polypeptides and peptides that comprise an epitope that upon exposure to the immune system of a vertebrate, either alone or in the presence of a molecule designed to augment the immune response, provokes a humoral immune response and/or a cellular immune response. Immunogenic polypeptides and peptides can be administrated for protection against viral, bacterial, parasitic diseases, or for modulation of the effects of autoimmune and allergic disease, or for control of various cancers (Wilson et al., (eds.) Harrison's Principles of Internal Medicine, 12th ed., 1991, McGraw-Hill, Inc., New York, pgs. 472-8. Austen et al. (eds.) Therapeutic Immunology, 1996, Blackwell Science, Cambridge, Mass., for a review of cytokines (pgs. 229-279), anti-idiotype-based therapies (pgs. 363-371), immunotherapy of allergic disease (pgs. 372-384), and vaccines and peptide therapy (pgs. 419-27; 441-450).

Examples of such polypeptides and peptides include, but are not limited to, polypeptides and peptides of viruses (e.g., viral envelope proteins, glycoproteins, surface antigens, etc.) such as influenza (e.g., influenza hemagglutinin of influenza types A, B, or C), herpes virus (HSV-1, HSV-2, EBV, varicella-zoster (chickenpox), and CMV), measles, mumps, rubella, polio, hepatitis viruses e.g. hepatitis A, B, and C surface antigene, RSV, papilloma virus, rabies, rotavirus, St. Louis encephalitis, HIV, FeLV, lymphocytic choriomeningitis, western equine encephalitis, and other viruses, particularly those pathogenic to humans and non-human animals, particularly non-human livestock and other domesticated animals. Examples also include polypeptides from pathogenic bacteria (e.g., bacterial surface proteins or toxins (e.g., diphtheria toxin, tetanus toxin, and toxins of Staphylococci, Yersiniae spp, Shigella spp. (e.g., S. dysenteriae, S. flexneri, S. boydii, and S. sonnei) enteropathogenic organisms such as Cholera spp, Neisseria spp. (e.g., N. meningitidis, N. gonohorroeae), Mycobacterium spp. (e.g. M. tuberculosis), Haemophilus spp. (e.g., H. influenzae, especially type b), Bordetella spp. (e.g., B. pertussis), Streptococcus spp., (e.g., S. pneumoniae, esp. group B strep), Mycoplasma spp. (e.g., M. pulmonis), Leishmania spp., Legionella spp., Chlamydia spp., Salmonella spp. (e.g., S. typhi), species of enteropathogenic Escherichia coli (e.g., EPEC, EIEC, and EHEC). Further examples also include polypeptides derived from pathogenic parasites, such as Plasmodium spp. (e.g., species associated with malaria), nematodes, cestodes, schistosomes, Trichomonas spp., Entamoeba spp., Ascaris spp.

More preferably, the transgenic Aquatic invertebrates of the present invention can be used as an oral vaccine or immuno-stumilator that has advantages such as convenience of administration and no need for purification or specialized technique for administration (see U.S. Pat. No. 5,723,755).

Production of Anti-Microbial Polypeptides and Peptides

The transgenic aquatic invertebrates of the present invention are useful in the preparation of anti-microbial polypeptides and polypeptides (see Zasloff, Curr. Opin. Immunol. 4: 3-7, 1992; Gudmundsson, et al., 1995. Proc. Natl. Acad. Sci. USA 92: 7085-7089, 1995, Valore et al., J. Clin. Investig. 101:1633-1642, 1998.)

Antimicrobial peptides useful in the methods of the present invention include, but are not limited to, broad spectrum antimicrobial peptides such as protegrin, magainins, dermaseptins, PGLa or XPF peptides, adrenoregulins, BPI protein and peptides, caeruleins, performs, insect defensins or sapecins, rabbit or human cationic antimicrobial peptides (CAP-18), porcine myeloid antibacterial peptides (PMAP), aibellins, acerins, brevenins, esculentins, lactoferrins, cecropin-mellitin hybrids (CEMA peptides), bombenins, tachyplesins, polyphemusins, human alpha and beta defensins, fish pleurocidin, cecropin B or synthetic lytic peptides such as SB-37, Shiva I through X, or Manitou.

Production of other Heteroloaous Polypeptides and Peptides

In addition to the production of immunogenic, therapeutic, and anti-microbial polypeptides and peptides, the present invention is also directed to the transgenic aquatic invertebrates that can be used as edible food sources or feeding materials for humans, livestock, poultry, and aquaculture without the necessity to purify the protein of interest. Human beings require a supply of essential amino acids for a well-balanced metabolism. The body cannot synthesize these amino acids by itself and, therefore, suitable quantities of these amino acids must be taken in by way of balanced nutrition. Nutritional deficiency in one or more of the essential amino acids leads to metabolic disturbances, e.g., hyperlipidemia, diabetes mellitus, etc., and accompanying hypertension. These disturbances are also a cause of diet-related overweight.

Therefore, in a balanced diet, it must be ensured that the essential amino acids are present in food in sufficient quantities and in correct proportion. This can be achieved, for example, by consuming animal protein such as fish, meat, sausage, cheese, and so forth. It is disadvantageous in this regard that, in addition to the proteins, large quantities of animal fat are also usually absorbed; this is undesirable especially for overweight persons. Therefore, it is the objective of the present invention to provide a method for the production of an easily digestible, protein-rich food source using the methods described herein. The use of transgenic aquatic invertebrates allows for the easy and inexpensive production of polypeptides and peptides, which have the added advantage of also expressing a transgenic polypeptide or peptide that is beneficial to the health of the organism consuming the transgenic aquatic invertebrate. Examples of such polypeptides and peptides include those already described herein, i.e., therapeutic polypeptides and peptides, immunogenic polypeptides and peptides, and anti-microbial polypeptides and peptides.

Other embodiments of the present invention include production of industrial enzymes, protease, lipases, chitinases, ligninases and other polypeptides and peptides of interest in research.

Purification of Gene Products from Transgenic Aquatic Invertebrates

Aquatic invertebrates such as adult Brine shrimp have a high protein content and non-calcified exoskeleton allowing for the easy purification of the polypeptides and peptides of interest using conventional physical, chemical, and biochemical purification approaches. Examples of purification steps in the present invention include, but are not limited to, homogenization, filtration with and without a filtration aid (e.g. dead-end filtration, filter presser, macrofiltration, microfiltration, ultrafiltration etc), centrifugation, ultracentrifugation, protein pi precipitation by adjusting solution pH with acids and bases, salt precipitation (e.g. ammonium sulfate precipitation), desalting, heat treatment for heat tolerance polypeptides, dialysis, gel filtration (e.g. Sephadex, Superdex, Superose, Sephacryl columns), affinity binding and elution (e.g. tag specific binding, metal, calmodulin benzamidine, glutathione, gelatin, lectin, nucleotide and nucleic acid, ConA etc.), immune binding and elution (e.g. polyclone and monoclone antibodies, protein A, protein G, etc.), FPLC and HPLC chromatography with ion exchange resin binding and elution (e.g. P11, DEAE, SP-Sepharose, Q-Sepharose, Monobeads etc.), with chromatofocusing (e.g. Mono P), with hydrophobic interaction (e.g. Phenyl Sepharose, Butyl/Octyl Sepharose, Alkyl Superose, RESOUCE columns), with reversed phase (Sephasil Protein C4/C8/C18 columns, μRPC C2/C18, RESOURCE RPC columns etc), electrophoresis (e.g. SDS-PAGE, native PAGE, semi-native PAGE, agrose electrophores, chromatofocusing electrophoresis), protein defolding and refolding (e.g. with urea, guanidinium HCl etc), drying (e.g. air drying, vacuum drying, fluid-air-bed drying, spray drying, lyophilization etc.). Not that an additional advantage of the present invention is that the protein may not need purifying at all, and may be available for use while still associated with the transgenic aquatic organism.

Extraction of DNA from Transgenic Aquatic Invertebrates

DNA isolation from aquatic invertebrates may be performed by either adapting methodologies designed for isolation of DNA from animal tissues with conventional organic solvents extraction or with commercially available purification kits for animal tissue (e.g. from Qiagen, Chatworth, Calif.). For DNA isolated for southern blot, aquatic invertebrates or decapsulated Artemia shrimp cysts could be homogenized or processed through several cycles of freeze/thawing. The tissues are lysed in the presence of sodium dodecyl sulfate (SDS), proteinase-K, and guanidinium HCl. The extracts are either extracted with chloroform followed by ethanol precipitation or the extracts are passed over a column matrix than binds double strained DNA and washed several times with a buffered solution to remove any contaminating proteins and lipids. The DNA can then be eluted from the column with water. DNA can be quantified with Agrose gel electrophoresis or with optical density measured at 260 nm.

DNA used for PCR amplification of transgenes in transgenic aquatic invertebrates can be performed with quick isolation methods, e.g. one or two of legs could be careful removed from an adult Brine shrimp (returning the living Brine shrimp back to the tank and it would still be able survive), the detached tissue is crushed in 20-50 microliter of 0.25 M NaOH, and boiled for 20 -60 seconds. After centrifugation at 12,000 rpm for 3-5 minutes, 2 μl of the supernatant is used as a PCR template. Similar methods can be used for the detection of transgenes in other aquatic invertebrates.

In addition to the methods outlined herein, one of skill in the art would appreciate that there are additional techniques that can be used in order to extract and purify DNA.

Detection of the Transgene with PCR

To determine if a transgenic aquatic invertebrate contains the appropriate gene of interest, PCR can be performed on either genomic DNA or cDNA sample derived from the aquatic invertebrate. The PCR amplification reaction is optimized based on multiple factors, including DNA quantity and quality, primers selection, size of the DNA fragment to be amplified, annealing temperature, and buffer concentration (See Griffin & Griffin (Eds.) 1994. PCR Technology: Current Innovations. CRC Press, Boca Raton, Fla.). To ensure specific detection of the transgene, a primer pair is designed to specifically amplify the gene of interest. An optimal region to amplify is normally between100 to 500 bp. PCR is normally performed for 25 to 35 cycles of amplification. An example of reaction is as follows: reaction buffer (10 mM Tris, pH 9.2; 1.5 mM MgCl₂; 75 mM KCl; 0.02% Tween-20; 10 μg/ml BSA), 200 μM each of each dATP, dCTP, dGTP and dTTP, 2 μM each primer and 1 μg of template DNA and 1.5 units of Taq polymerase. Samples are heated to 95° C. for 2 min and then the reaction mixtures are cycled in a DNA Thermal Cycler 25-35 times at 94° C. for 1 min, 60° C. for 1 min, and 72° C. for 1 min with a final extension step at 72° C. for 10 min. PCR products are resolved on a 2% agarose gel in the presence of ethidium bromide (EtBr, 10 ng/ml final concentration in the gel) and using 1XTAE buffer.

Advantages of some aquatic invertebrates, such as Artemia shrimp, is that Artemia shrimp can regenerate certain body parts when lost, e.g. one or two legs can be removed and used in the analysis transformation efficacy. PCR amplification for the presence of transgenic genes provides for an easy and routine method for determining the presence of the gene.

RNA Isolation Methods

RNA isolation from Brine shrimps may be performed by either adapting methodologies designed for isolation of RNA from animal tissues with conventional organic solvents extraction (Molecular Cloning second edition) or with commercially available purification kits from animal tissues (e.g. from Qiagen, Chatworth, Calif.). In general, tissues of aquatic invertebrates could be lysed in presence of protein denature agents such as guanidinum HCl. The RNA is extracted with chloroform and precipitated with RNA specific precipitants such as lithium chloride, sodium acetate, isopropanol and ethanol. DNA contamination can be removed by digestion with DNase. Additional methods from RNA isolation involve passing the extracts over a column matrix that binds single strand RNA under specific solvents condition and other contaminants including double strand DNA are washed away with buffered solutions. The RNA is eluted with RNase-free buffer from the column. RNA can be quantified with either Agarose or polyacrymid gel electrophoresis or with optical density at 260 nm.

Reverse Transcription Polymerase Chain Reaction (RT-PCR)

In order to confirm that the inserted genes are properly transcribed into mRNA, RT-PCR is conducted with total RNA isolated from the transgenic aquatic invertebrate. RT-PCR is a method in which PCR is applied in conjunction with reverse transcription. Typically, the RNA is extracted from the aquatic invertebrate, and reverse transcribed to produce cDNA molecules. The first DNA strand can be synthesized using the following methods: mRNA is reverse transcribed into cDNA using the reverse transcriptase, moloney murine leukemia virus (MMLV), in a reaction mixture containing 100 mM Tris-HCl, 50 mM KCl, 5 mM MgC12 and 1 mM dNTP. The reverse transcriptase is inactivated at 99° C. for 5 minutes, and then PCR is performed under the conditions of pre-denaturation at 94° C. for 2 minutes in a reaction solution containing 200 μl of the PCR buffer solution (2 mM MgCl2, 10 mM Tris, pH 8.3, 50 mM KCl) supplemented with 0.6 mM of both primers. The cycle of denaturation (94° C. for 1.5 minutes), annealing (55 to 60° C. 1 for 0.5 minutes) and extension (72° C. for 2.5 minutes) are repeated 25-30 times and the amplified DNA fragments corresponding to the gene of interest are separated on a 2% agarose gel using electrophoresis.

Immuno-Detection of Gene Products

Generally, a transgenic aquatic invertebrate can be assayed for the presence of the expressed gene products. Appropriate assays for detecting a gene product of interest in tissue are well known to those of skill in the art. For example, using an antibody that specifically binds the expressed gene product in an ELISA assay one can determine if the aquatic invertebrate has been successfully transduced with the gene of interest. This assay can be performed either qualitatively or quantitatively. The ELISA assay, as well as other immunological assays for detecting a protein in a sample, are described in Antibodies: A Laboratory Manual (1988, Harlow and Lane, eds. Cold Spring Harbor Laboratory, Cold Spring Harbor, N.Y.).

Pharmaceutical Compositions

In additional embodiments, the present invention concerns formulation of one or more of the polypeptides or peptides disclosed herein and a pharmaceutically-acceptable carrier for administration to a cell or an animal, either alone, or in combination with one or more other modalities of therapy.

Pharmaceutical compositions of this invention comprise either a transgenic invertebrate described herein, wherein the transgenic invertebrate comprises a polynucleotide sequence encoding a heterologous protein, or a heterologous polypeptide or peptide which has been produced and isolated from said transgenic invertebrate, and pharmaceutically acceptable salts thereof, with any pharmaceutically acceptable carrier, adjuvant or vehicle (hereinafter collectively referred to as “pharmaceutically-acceptable carriers”). Pharmaceutically acceptable carriers, adjuvants and vehicles that may be used in the pharmaceutical compositions of this invention include, but are not limited to, ion exchange, alumina, aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene-polyoxypropylene-block polymers, polyethylene glycol and wool fat, and the like.

The pharmaceutical compositions of this invention may be administered orally, parenterally, by inhalation spray, topically, rectally, nasally, buccally, vaginally or by an implanted reservoir. Oral and parenteral administration are preferred. The term “parenteral” as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to, capsules, tablets, and aqueous suspensions and solutions. In the case of tablets for oral use, “carriers” that are commonly used include lactose and corn starch. Lubricating agents, such as magnesium stearate, are also typically added. For oral administration in capsule form useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature. Such materials include, but are not limited to, cocoa butter, beeswax and polyethylene glycols.

Topical administration of the pharmaceutical compositions of this invention is especially useful when the desired treatment involves areas or organs readily accessible to topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene, polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-applied transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The compounds of this invention can also be administered in combination with immunomodulators (e.g., bropirimine, anti-human alpha interferon antibody, IL-2, GM-CSF, methionine enkephalin, interferon alpha, diethyldithiocarbamate, tumor necrosis factor, naltrexons and rEPO) or with prostaglandins, to prevent or combat IL-1-mediated disease symptoms such as inflammation.

When the compounds of this invention are administered in combination therapies with other agents, they may be administered sequentially or concurrently to the patient. Alternatively, pharmaceutical compositions according to this invention may be comprised of a combination of a compound of Formula I and another therapeutic or prophylactic agent mentioned above.

The following examples are offered by way of illustration and not by way of limitation.

### EXAMPLES

### Example 1

### PRODUCTION OF BRINE SHRIMP EGGS OR CYSTS

Adult female Brine shrimp are mechanically separated based on the visual identification of morphological differences between the male and female shrimp. Once the female shrimp are identified, they are transferred into a separate container and cultured in solution that contains 2-15% of NaCl, 1 to 10 ppm K₂HP0₄. NaHCO₃ is used to adjust the solution pH to 8.0-9.0. The temperature of the culture is maintained at 20 to 30° C., and continually aerated using an aqua air pump, with an oxygen probe used to monitor the oxygen level. The Brine shrimp are fed with bacteria, yeast, microalgea, wheat flavor, soybean flavor, whey or even fresh chicken manure. After the culture reaches a density of between 500 to 5000 shrimps per liter, NaCl can be added to adjust the saline concentration to higher than 6.5% or the oxygen level can be reduced to below to 2 mg per liter. These changes to the culture conditions stimulate the female Brine shrimp to produce dormant eggs of cysts. Using a high saline concentration has the added advantage in causing the cysts to float on the surface of the culture, allowing their easy harvest using a fine net. Alternatively, the female Brine shrimp can be stimulated to produce cysts or eggs via the reduction of their food supply. The cysts or eggs can then be air-dried and stored for future use.

### Example 2

Preparation of Decapsulated Eggs or Cysts for Gene Transformation and Hatching

The best way to introduce heterologous nucleic acids into Brine shrimp, is to start from a Brine shrimp egg or cyst. The eggs or cysts are surrounded by a very hard shell and have to be decapsulated before used for gene transformation. Dried cysts or eggs are first hydrated into a spherical shape with distilled water or sea water at room temperature for 30 to 90 minutes, longer incubation times are not recommended as the cysts or eggs will have started to resume their metabolisms therefore likely would not survive the decapsulation procedure. The decapasultion process uses the following steps: re-suspending one gram of eggs or cysts in 4.67 ml of water or sea water, followed by the slow addition of 0.33 ml of 10 N sodium hydroxide (40% NaOH). The solution must be stirred well and the reaction starts by the addition of 10 mls of house bleach (Clorox with 6% sodium hypochlorite). The solution should be kept at a temperature range of 20 to 30° C. The resulting mixture is stirred until the color of solution turns from dark brown to gray, to white and then to bright orange. The whole process usually takes less than 5 minutes, and is somewhat dependent on the mixtures temperature and the eggs and cysts themselves. Once the color change has occurred, immediately filter the solution through nets and rinse the eggs or cysts with fresh water to remove the chlorine. The eggs and cysts are washed in 0.1% sodium thiosulfate and 0.5% acetic acid solution for one minute to neutralize the reaction and completely remove the chlorine from the solution. The cysts are then re-washed well with fresh or salt water. The resulted decapsulated eggs and cysts can be used immediately for gene transfer or can be stored in a refrigerator for up to week for hatching.

### Example 3

Gene Transfer into Brine Shrimp Eggs or Cysts

This example describes the process by which heterologous genes are expressed in the Brine shrimp eggs and cysts isolated in Example 1. A linear gene expression cassette, containing a eukaryotic promoter, a desired gene sequence, a polyA sequence and a transcription terminator is isolated by digestion with proper restriction enzyme from a cloned vector that contains the expression cassette. The desired DNA is then purified from agarose using either electrophoresis and/or column purification. Purified DNA is dissolved in pure water at suitable a concentration. A Bio-Red Gene Pulser is used in the present invention in order to introduce the DNA of interest into the Brine shrimp cyst. In order to perform this, 500 to 1000 cysts are re-suspend in 5 ml of autoclaved or sterilized sea water in a 9 mm petri dish and the appropriate amount of DNA is added, followed by incubation at 4° C. for 30 minutes. Approximately 0.8 mls of the cyst mixture is transferred to the Gene Pulser Cuvette. With a 0.8 ml volume. and 0.4 cm distance, electroportation parameters with Capacitance Extender are set at the voltage of 200-400 V and capacity at 125 to 960 μF. After electroportation, the cysts are incubated at room temperature in a petri-dish and placed on a slow rotation shaker. Following 24 to 36 hours of incubation, the Brine shrimp will hatch and the hatched Brine shrimp can be transferred into a container containing 100 to 500 ml seawater with aeration. The Brine shrimp are grown under the appropriate conditions and at around 12 to 14 days post-culture, single female Brine shrimp are separated into individual containers and grown under optimal conditions to allow them to reproduce via parthenogenesis. The transduced Brine shrimps are analyzed with maker gene expression, gene product immune detection, PCR or any other gene analysis methods.

### Example 4

Total RNA Isolation from Transgenic Brine Shrimp

Total RNA can be isolated from the transgenic Brine shrimp using the tri-reagent method or commercially available animal tissue purification kit. In order to perform these experiments, approximately 0.2 g of the tissue is ground into a fine powder in liquid nitrogen, mixed with 1 ml of tri-reagent, agitated for 10 seconds, and placed on ice for 15 minutes. 0.2 ml of chloroform is added to the mixture, the reaction allowed to proceed for 15 minutes, followed by centrifugation at 3,000 rpm at 4° C. for 20 minutes. 1 ml of isopropanol is added to the supernatant to precipitate the RNA. The resulting mixture is allowed to incubate for 10 minutes followed by further centrifuged at 10,000 xg for 20 minutes. The resulting RNA pellet is washed with 75% ethanol, resuspended in sterile DEPC-treated water, and stored at −70° C. The optical density is measured at 260 nm and 280 nm.

### Example 4

Immune Detection of a Transgenic Gene Product in Brine Shrimp

Protein isolated from the Brine shrimp is diluted with PBS and bonded to an EIISA plate at 4° C. overnight. The wells are washed with PBST (1% Triton in PBS) and blocked with 5% PBSA (5% BSA in PBS). After further washing of the wells with PBST twice, the protein extract in each well is allowed to react with an antibody diluted in 3% PBSA at room temperature for 2 hours. The well is washed again with PBST twice and IgA-horseradish peroxidase conjugate diluted in 3% PBSA (1:1,000) is added. The reaction is performed at room temperature for 1.5 hours. The well is washed with PBST twice, and developed by ECL solution A and B from Amersham Life Science.

### Example 5

Analysis of Protein by SDS-Page and Western Blot

The transgenic Brine shrimp tissue is homogenized in cold proper extraction buffer (e.g. 10 mM Tris, pH 6.5 to 8.5, 20 to 500 mM NaCl, 0.001 to 1% Triton X100), and 5 to 25 μl of the obtained fraction of protein is mixed with 1 to 5 μl of a 6 X SDS buffer solution (0.35 M Tris-Cl, pH 6.8, 10% SDS, 30% glycerol, 9.3% DDT), boiled at 100° C. for 5 minutes, and electrophoresed on 4 to 20% polyacrylamide gel at voltage of 125 V. After the marker dye reaches the bottom of the gel, the gel is silver stained in order to detect the band corresponding to the protein of interest.

The protein is transferred onto a nitrocellulose membrane and nonspecific binding is blocked using skim milk (3% skim milk in PBS) by incubation for 1.5 hours. The membrane bound protein is washed with a PBST buffer solution (1% Triton X-100 in PBS) and allowed to react with the primary antibody which is targeted against the protein of interest.

Following the washing of the membrane with PBST (1% Triton X-100 in PBS), the second antibody (Anti-mouse or rabbit lg, horseradish peroxidase linked whole antibody), which is diluted with the blocking solution is reacted with the primary antibody bound to the protein at room temperature for 50 minutes.

After washing with PBST (1% Triton X-100 in PBS), the membrane is developed with ECL solution from Amersham Life Science, to detect the antigen-specific proteins.

### Example 6

Isolation and Purification of a Transgenic Protein from a Brine Shrimp Culture

The transgenic Brine shrimp is harvested, washed with cold fresh water several times and homogenized in cold extraction buffer (e.g. 10 mM Tris, pH 6.5 to 8.5, 20 to 500 mM NaCl, 0.01 to 100 mM EDTA, 0.001 to 1% Triton X100, protease inhibitors). The solution is centrifuged and the pellet is solubilized with either 8 M urea or 6 M guanidine HCl containing 10 mM Tris pH 8.0 plus 10 mM imidazole. The solubilized protein is added to 5 ml of nickel-chelate resin (Qiagen) and incubated for 45 min to 1 hour at room temperature with continuous agitation. After incubation, the resin and protein mixture is poured through a disposable column and the flow through is collected. The column is washed with 10-20 column volumes of the solubilization buffer. The protein is eluted from the column using 8 M urea, 10 mM tris pH 8.0 and 300 mM imidazole and collected in 3 ml fractions. A SDS-PAGE gel is run to determine which fractions to pool for further purification. As a final purification step, a strong anion exchange resin such as Hi-Prep Q (Biorad) is equilibrated with the appropriate buffer and the pooled fractions from above are loaded onto the column. Each protein is eluted off of the column with an increasing salt gradient. Fractions are collected as the column is run and another SDS-PAGE gel is run to determine which fractions from the column to pool. The pooled fractions are dialyzed against 10 mM Tris pH 8.0. This material is then evaluated for acceptable purity as determined by SDS-PAGE or HPLC, concentration as determined by Lowry assay or Amino Acid Analysis, identity as determined by amino terminal protein sequence, and endotoxin level as determined by the Limulus (LAL) assay. The protein is frozen after filtration through a 0.22 micron filter.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-95. (Canceled)

96. A transgenic aquatic invertebrate comprising a polynucleotide sequence, wherein said polynucleotide sequence encodes a heterologous polypeptide or peptide.

97. An aquatic invertebrate bioreactor for the large-scale production of a heterologous polypeptide or peptide, said bioreactor comprising an aquatic invertebrate comprising a polynucleotide sequence, wherein said polynucleotide sequence encodes a heterologous polypeptide or peptide.

98. A method for producing a heterologous polypeptide or peptide in an aquatic invertebrate comprising the steps of:
(a) producing a construct comprising a polynucleotide sequence encoding for a heterologous polypeptide or peptide; and
(b) inserting said construct into the genome of an aquatic invertebrate, such that expression of said polynucleotide sequence is under the control of a regulatory region of DNA that regulates expression of said heterologous polypeptide or peptide.

99. A method for producing an Artemia cyst comprising a polynucleotide sequence encoding a heterologous polypeptide or peptide, comprising the steps of:
(a) hydrating a cyst;
(b) decapsulating said hydrated cyst;
(c) producing a construct comprising a polynucleotide sequence encoding for a heterologous polypeptide or peptide; and
(d) inserting said construct into the genome of said decapsulated cyst such that the expression of said polynucleotide sequence encoding said heterologous protein is under the control of a regulatory region of DNA that regulates the expression of said heterologous polypeptide or peptide.

100. A method for generating an Artemia shrimp cyst, comprising growing an Artemia shrimp under conditions suitable for inducing said Artemia shrimp to produce resting cysts.

101. The method of claim 100, wherein said conditions are selected from the group consisting of: high salinity, low oxygen level, and chronic food shortage.

102. The transgenic aquatic invertebrate of claim 96, wherein said transgenic aquatic invertebrate is a crustacean.

103. The transgenic aquatic invertebrate of claim 102, wherein said crustacean is selected from the group consisting of: isopods, copepods, branchiopods, and decapods.

104. The transgenic aquatic invertebrate of claim 103, wherein said decapod is selected from the group consisting of: crabs, lobsters, crayfish, and shrimp.

105. The transgenic aquatic invertebrate of claim 103, wherein said branchiopod is selected from the group consisting of: fairy shrimp, Brine shrimp, daphnia, clam shrimp, and tadpole shrimp.

106. The transgenic aquatic invertebrate of claim 96, wherein said transgenic aquatic invertebrate is an Artemia shrimp.

107. The transgenic aquatic invertebrate of claim 96, wherein said polynucleotide sequence encodes a therapeutic polypeptide or protein.

108. The transgenic aquatic invertebrate of claim 107, wherein said therapeutic polypeptide or protein is selected from the group consisting of: human calcitonin, mouse calcitonin, salmon calcitonin, insulin, growth hormone, growth hormone releasing factor, somatostatin, thyrotropin, tissue-type plasminogen activator, vasopressin, human cholesterol hydolase, lipocortin, coagulation factors VIII and IX, thrombopoietin, alpha-antitrypsin, erthropoietin, urokinase, IFN-gamma, IL-1, IL-2, IL-5, IL10, IL-11, IL-12, IL-18, tumor necrosis factor, colony stimulating factor, GM-CSF, and human cholesterol hydrolase.

109. The transgenic aquatic invertebrate of claim 96, wherein said polynucleotide sequence encodes an immunogenic polypeptide or peptide.

110. The transgenic aquatic invertebrate of claim 109, wherein said immunogenic polypeptide or peptide selected from or derived from the group consisting of: influenza types A, influenza B, influenza C, HSV-1, HSV-2, EBV, varicella-zoster, CMV, measles, mumps, rubella, polio, hepatitis A, hepatitis B, hepatitis C, RSV, papilloma virus, rabies, rotavirus, St. Louis encephalitis, HIV, FeLV, lymphocytic choriomeningitis, western equine encephalitis, other viruses, diphtheria toxin, tetanus toxin, toxins of Staphylococci, Yersiniae, Shigella, S. dysenteriae, S. flexneri, S. boydii, S. sonnei, Cholera, Neisseria, N. meningitidis, N. gonohorroeae, Mycobacterium, M. tuberculosis, Haemophilus, H. influenzae, Bordetella, B. pertussis, Streptococcus, S. pneumoniae, Mycoplasma, M. pulmonis, Leishmania, Legionella., Chlamydia., Salmonella, S. typhi, EPEC, EIEC, EHEC, Plasmodium, nematodes, cestodes, schistosomes, Trichomonas, Entamoeba, and Ascaris.

111. The transgenic aquatic invertebrate of claim 96, wherein said polynucleotide sequence encodes an anti-microbial polypeptide or peptide.

112. The transgenic aquatic invertebrate of claim 111, wherein said anti-microbial polypeptide or peptide is selected from the group consisting of: protegrin, magainins, dermaseptins, PGLA peptides, XPF peptides, adrenoregulins, BPI protein, BPI peptides, caeruleins, performs, insect defensins, insect sapecins, rabbit cationic antimicrobial peptides, human cationic antimicrobial peptides, porcine myeloid antibacterial peptides, aibellins, acerins, brevenins, esculentins, lactoferrins, cecropin-mellitin hybrids, bombenins, tachyplesins, polyphemusins, human alpha defensins, human beta defensins, fish pleurocidin, cecropin B, the synthetic lytic peptide SB-37, Shiva I through X, and Manitou.

113. The aquatic invertebrate of claim 96, wherein said polynucleotide sequence encodes a polypeptide or peptide suitable as a food source for human, animals, or aquatic creatures.

114. The aquatic invertebrate of claim 96, wherein said polynucleotide sequence encodes a polypeptide or peptide suitable as an industrial enzyme, protease, lipases, chitinases, ligninases or other polypeptides or peptides of interest in research.
